(19) United States
(12) Patent Application Publication
(10) Pub. No.: US 2026/0250618 A1
(43) Pub. Date: Aug. 27, 2026

(54) FOAM BREAKER DEVICE AND METHODS OF USE

(71) Applicant: Life Technologies Corporation, Carlsbad, CA (US)

(72) Inventors: Jason D. Brown, Logan, UT (US); Nephi D. Jones, Newton, UT (US)

(21) Appl. No.: 18/995,346

(22) PCT Filed: Jul. 17, 2023
(86) PCT No.: PCT/US2023/027924

§ 371 Date: Jan. 16, 2025

Related U.S. Application Data
(60) Provisional application No. 63/390,278, filed on Jul. 18, 2022.

(51) Int. Cl. C12M 1/21; C12M 1/00; C12M 3/00

(52) U.S. Cl. CPC C12M 41/02; C12M 23/26; C12M 23/46

(57) ABSTRACT

Described herein are various methods and embodiments of a fluid processing system and foam breaker devices for at least reducing foam formation in a container of the fluid processing system. In some embodiments, the foam breaker device includes a mounting hub extending along a longitudinal axis of the foam breaker device. The mounting hub can include a coupling feature for coupling the foam breaker device to a drive shaft of the fluid processing system. The foam breaker device can also include a wall structure extending circumferentially from the mounting hub at an angle relative to the longitudinal axis of the foam breaker device. The wall structure can cause a reduction in the foam volume as a result of at least a part of the wall structure contacting the foam volume.

### CROSS-REFERENCE TO RELATED APPLICATIONS

This application claims the benefit of U.S. Provisional Application No. 63/390,278, filed on Jul. 18, 2022, which is incorporated herein by specific reference.

### BACKGROUND

The biopharmaceutical industry uses a broad range of mixing systems for a variety of processes such as in the preparation of media and buffers and in the growing, mixing and suspension of cells and microorganisms. Some conventional mixing systems, including bioproduction equipment (e.g., bioreactors and fermenters), comprise a flexible bag disposed within a rigid support housing. An impeller is disposed within the flexible bag and is coupled with a drive shaft projecting into the bag. Rotation of the drive shaft and impeller facilitates mixing and/or suspension of a liquid contained within the flexible bag.

For example, the growing, mixing, and suspension of cells and microorganisms in a liquid contained in the flexible bag can generate a foam layer within the flexible bag above the liquid. Excessive foam inside the flexible bag and bioreactor can clog filters associated with the mixing system, interfere with oxygen transfer at the liquid surface, result in unwanted buildup of gas (e.g., ammonia, carbon dioxide), and/or cause the flexible bag to implode and compromise a sanitary seal. For example, filters becoming clogged due to foam can be problematic as clearing the filters can interfere with processing of the liquid. Additionally, compromising the sanitary seal can contaminate the liquid and result in disposal of the liquid. Foam can also entrain liquid additions such as acid and base buffers critical for pH control or entrain solutions such as nutrient, carbon source, or reagents critical for success of the process. As such, excessive foam can disrupt the growing, mixing, and suspension of cells and microorganisms or homogenous mixing of fluid processing.

Currently, the amount of foam and/or the thickness of the foam layer can be controlled using anti-foam solutions. Such anti-foam solutions present their own set of challenges, including reducing oxygen transfer and complicating downstream purification of the liquid. For example, if anti-foam solution is added to the liquid, the anti-foam solution must be removed from the final product. This anti-foam purification process can be detrimental to the quality of the final product and present extra processing steps. For example, anti-foam solution can be detrimental to the growing, mixing, and suspension of cells and microorganisms. As such, the amount of anti-foam solution applied to the foam and liquid should be minimized. Accordingly, there are several technical and logistical challenges associated with foam formation in bioproduction equipment, as well as in other liquid mixing systems.

### SUMMARY OF THE DISCLOSURE

It is understood that each independent aspect recited herein may include any of the features, options, and possibilities recited in association with the other independent aspects set forth above or as recited elsewhere within this document.

Example systems and methods for a foam breaker device are herein disclosed. An example foam breaker device can include a mounting hub extending along a first axis of the foam breaker device. The mounting hub can be configured to rotatably couple to a drive motor assembly. Further, a wall structure extends circumferentially from the mounting hub at an angle relative to the first axis of the foam breaker device. The wall structure can cause a reduction in foam volume when at least a part of the wall structure contacts the foam volume. The wall structure can extend at an angle approximately between 10 degrees and 90 degrees. The wall structure can be formed of a flexible material, a foldable material, a hydrophilic material, and/or a hydrophobic material. An inner wall surface or an outer wall surface of the wall structure can have a macro texture or micro texture. The wall structure can include two or more overlapping fin structures or a plurality of radially extending bristle structures. The wall structure can be adjustable between an extended configuration having a first outer diameter and a collapsed configuration having a second outer diameter that can be smaller than the first outer diameter. The wall structure can include a through hole positioned adjacent to the mounting hub for allowing one or more of a liquid, like a cell culture medium, and gas to pass through the hole. The mounting hub can include a coupling feature configured to couple the mounting hub to a first shaft. A passageway can extend along the mounting hub and can be configured to receive the first shaft. The coupling feature can be shaped to have a sliding translational engagement with the first shaft. The coupling feature can be rotationally fixed relative to the first shaft. Alternatively, the foam breaker device can be coupled to the first shaft such that rotation of the first shaft causes the foam breaker device to rotate about the first axis. The foam breaker device can rotate at a speed of approximately 50 rotations per minute (RPMs) to approximately 1200 RPMs.

In various embodiments, a fluid processing system is provided that includes a container having an inner chamber configured to contain a liquid, and a foam breaker device. A drive shaft extends into the inner chamber of the container for assisting with mixing the liquid, and the foam breaker device can be coupled to the drive shaft. The foam breaker device can also be positioned along a port in fluid communication with an inner chamber of the container. The drive shaft can include one or more of a flexible drive shaft, a rigid drive shaft, a foldable drive shaft, and a drive shaft having a ladder and/or helical configuration. The foam breaker device can be coupled to the drive shaft in a first orientation relative to the drive shaft, and the first orientation includes an inner surface of the foam breaker device directed toward a distal end of the drive shaft and/or a lower end of the container. Additionally, the foam breaker device can be coupled to the drive shaft in a second orientation relative to the drive shaft, and the second orientation including the inner surface of the foam breaker device directed toward a proximal end of the drive shaft and/or upper end of the container. The container can include one or more of a bioreactor, a flexible bag, a condenser, a fermenter, a mixing chamber, and tubing.

In various embodiments, a method of processing a liquid in a fluid processing system is provided. A liquid can be received in an inner chamber of a container of the fluid processing system, and a drive shaft extending into the inner chamber can be rotated for mixing the liquid. Further, a foam breaker device can be coupled to the drive shaft and rotated to reduce the foam volume in the inner chamber. The liquid in the inner chamber can include one or more of human cells, microorganisms, bacteria, fungi, algae, plant cells, animal cells, protozoans, and nematodes. The container of the fluid processing system can include one or more of a bioreactor, a flexible bag, a condenser, a fermenter, a mixing chamber. A perimeter of the wall structure can include a diameter that can be greater than three-quarters of the inner diameter of the inner chamber of the container. A ratio of the diameter of the foam breaker device to the inner diameter of the inner chamber of the container can be 1:2.

In various embodiments, a method of assembling a fluid processing system is provided. A foam breaker device can be coupled to a drive shaft of a mixing system. The foam breaker device can include a mounting hub extending along a first axis of the foam breaker device, the mounting hub can have a coupling feature for coupling the foam breaker device to the drive shaft. A cone shaped wall can extend circumferentially from the mounting hub at an angle relative to the first axis of the foam breaker device, the cone shaped wall reducing the foam content when at least a part of the cone shaped wall contacts the foam. The coupling feature of the mounting hub can be coupled to the drive shaft to secure the foam breaker device at a position along the drive shaft. Optionally, the coupling feature can be rotationally fixed relative to the drive shaft when coupled thereto such that rotation of the drive shaft causes rotation of the foam breaker device. The coupling feature can have a square or hexagonal cross-section. Additionally, one or more foam breaker devices can be coupled to the drive shaft. The container can include one or more of a bioreactor, a flexible bag, a condenser, a fermenter, a mixing chamber, and a tubing. Optionally, the foam breaker device can be positioned along a port in fluid communication with the inner chamber of the container.

In various embodiments, a method of suppressing foam in a fluid processing system is provided. A drive shaft extends into an inner chamber containing a liquid. A foam breaker device can be coupled to the drive shaft and can be rotated to reduce and/or prevent a foam volume in the inner chamber.

In various embodiments, a method of processing a liquid in a fluid processing system is provided. A liquid can be received in an inner chamber of the container of the fluid processing system. A drive shaft extends into the inner chamber and can be rotated for mixing the liquid. A foam breaker device can be coupled to the drive shaft and rotated to reduce a foam volume in the inner chamber. Preferably, the foam breaker device can be positioned above the liquid.

In various embodiments, a method of reducing foam in a container of a bioprocessing system is provided. The system can include a foam breaker device having a mounting hub extending along a first axis of the foam breaker device. The mounting hub can be rotatably coupled to a drive motor assembly, and a wall structure extends circumferentially from the mounting hub. The foam breaker device can be rotated at a first speed to extend the foam breaker device from a first configuration to a second configuration. Then the foam breaker device can be rotated at a second speed such that the foam can be contacted by at least a portion of the wall structure of the foam breaker device to reduce foam in the container. The wall structure of the foam breaker device in the second configuration can be aligned with the first axis at an angle ranging between 10-90 degrees. The first speed can be less than the second speed and the second speed can range between 140-160 inches/second.

In various embodiments, a method of reducing foam in a container of a bioprocessing system is provided. The system can include a foam breaker device having a mounting hub extending along a first axis of the foam breaker device. The mounting hub can be rotatably coupled to a drive motor assembly, and a wall structure extends circumferentially from the mounting hub. The foam breaker can be held in a first configuration, then the form breaker can be released to extend the wall structure of the foam breaker device from the first configuration to a second configuration. The foam breaker device can be maintained at the second configuration and rotated to cause at least a portion of the wall structure of the foam breaker device to continuously contact the foam to reduce the foam in the container. The foam breaker device can include a memory metallic wire to substantially support the wall structure or be embedded in the wall structure.

In various embodiments, a foam breaker device for a fluid processing system is provided. The foam breaker device can include a mounting hub extending along a first axis of the foam breaker device. The mounting hub can be configured to rotatably couple to a drive motor assembly. Further, a plurality of wall structures can extend circumferentially from the mounting hub at an angle relative to the first axis of the foam breaker device. Each of the wall structures can cause a reduction in foam volume when at least a part of each of the wall structures contacts the foam volume.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a perspective view of a fluid processing system according to exemplary embodiments of the present disclosure.

FIG. 2 illustrates a perspective view of a container and drive motor assembly of the fluid processing system of FIG. 1 according to exemplary embodiments of the present disclosure;

FIG. 3 illustrates a perspective partially exploded view of the drive motor assembly and impeller assembly of FIG. 2;

FIG. 4A illustrates a side perspective view of an embodiment of a foam breaker device according to exemplary embodiments of the present disclosure;

FIG. 4B illustrates a top view of the foam breaker device of FIG. 4A;

FIG. 4C illustrates a side cross-section view of the foam breaker device of FIG. 4B taken along lines 4C-4C;

FIG. 4D illustrates a side perspective view of an embodiment of the foam breaker device of FIG. 4A including a collapsible cone shaped wall shown in a collapsed configuration;

FIG. 4E illustrates an end perspective view of the foam breaker device of FIG. 4D;

FIG. 4F illustrates a bottom view of the foam breaker device of FIG. 4D;

FIG. 4G illustrates a top view of the foam breaker device of FIG. 4D;

FIGS. 4H-4L illustrate top views of alternate embodiments of the foam breaker device of FIG. 4A;

FIG. 5A illustrates a side view of an embodiment of the foam breaker device coupled to a part of the impeller assembly and positioned in a container including a volume of foam;

FIG. 5B illustrates a side view of an embodiment of the foam breaker device coupled to the part of the impeller assembly and positioned in a container including a reduced volume of foam;

FIG. 5C illustrates a side view of an embodiment including four foam breaker devices coupled to a part of the impeller assembly;

FIG. 6A illustrates a side perspective view of an embodiment of three foam breaker devices coupled to a part of the impeller assembly;

FIG. 6B illustrates a side cross-section view of the three foam breaker devices coupled to the part of the impeller assembly of FIG. 6A;

FIG. 7A illustrates a top view of an embodiment of the foam breaker device;

FIG. 7B illustrates a side cross-section view of the foam breaker device of FIG. 7A taken along lines 7B-7B;

FIG. 8 illustrates a side-exploded view of an embodiment of three foam breaker devices and an impeller configured to couple to a second tubular connector of the impeller assembly;

FIG. 9 illustrates a perspective view of an embodiment of the container having a port including a tubing with an embodiment of the foam breaker device positioned therealong; and

FIG. 10A illustrates a perspective view of an embodiment of the container and fluid processing system including a foam breaker device and drive shaft having a ladder configuration;

FIG. 10B illustrates a perspective view of an alternate embodiment of the container and fluid processing system including a foam breaker device and drive shaft having a ladder configuration;

FIG. 11A illustrates a side view of an alternate embodiment of a foam breaker devices of FIG. 4A; and

FIG. 11B illustrates a side view of another alternate embodiment of a foam breaker devices of FIG. 4A.

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION

The present disclosure provides systems and methods for efficiently and effectively reducing a foam volume and/or foam formation in a part of a fluid processing system. For example, various embodiments of a foam breaker device are described that are configured to reduce a foam volume and/or foam formation in the fluid processing system. For example, foam can form in the fluid processing system as a result of mixing or processing a solution contained in the fluid processing system. Such foam formation can cause clogging of filters, which can damage such filters and/or make such filters ineffective (e.g., due to being clogged with foam). Other problems can arise as a result of foam formation, such as foam formation interfering with successful fluid processing. For example, foam formation can cause an unwanted buildup of a gas (e.g., ammonia, carbon dioxide) in the fluid processing system and/or interfere with oxygen transfer at a surface of the solution contained in the fluid processing system.

In some cases, an anti-foam solution can be added to a chamber of the fluid processing system to reduce a foam volume and/or foam formation. However, such anti-foam solution can interfere with some processes and can require additional processing steps to remove the anti-foam from the solution. As such, use of anti-foam solution can increase time and costs associated with fluid processing system processes.

Various embodiments of fluid processing systems and foam breaker devices are disclosed herein that can reduce time and costs associated with fluid processing system processes, such as by reducing foam volume and foam formation, as well as eliminating the need to use anti-foam all together or at least reducing the amount of anti-foam necessary to eliminate or reduce foam in the process.

For example, the present disclosure includes various embodiments of foam breaker devices for reducing a foam volume and/or foam formation in one or more parts of a fluid processing system. Fluid processing system disclosed herein can be, but are not limited to mixers, reactors, fermenters, bioprocess containers, filters, fluid storage containers, bubble traps, conduits, pumps, valves, or other bioproduction or process vessels used to process store, or flow biological fluids and/or biological components. For example, the fluid processing system can be configured for processing (e.g., mixing, sparging, reacting, fermenting, etc.) solutions and/or suspensions. In some embodiments the fluid processing systems can include fluid management systems, cell culture equipment, centrifuges, centrifugal separators, chromatography units, mixers, homogenizers, magnetic processing units, blood separating devices, biocomponent filtering devices, biocomponent agitators or any other device designed for growing, mixing or processing cells and/or other biological components. It is also appreciated that the fluid processing system can comprise any conventional type of bioreactor, fermenter, or cell culture device such as a stirred-tank reactor, rocker-type reactor, paddle mixer reactor, or the like. In some embodiments, the fluid processing system can include one or more impellers for mixing solutions and/or suspensions. In one embodiment, the processing systems can be bioreactors or fermenters used for culturing cells or microorganisms. By way of example and not by limitation, the disclosed systems can be used in culturing bacteria, fungi, algae, plant cells, animal cells, protozoans, nematodes, and the like. Furthermore, the foam breaker devices can reduce foam volume and/or foam formation during such culturing, thereby eliminating or at least reducing the need for adding anti-foam solution to the solution or suspension, as well as reducing processing disruptions and/or damage to the fluid processing system.

In some embodiments, the fluid processing system can accommodate cells and microorganisms that are aerobic or anaerobic and are adherent or non-adherent. The fluid processing system can also be used in association with the formation and/or treatment of solutions and/or suspensions that are not biological but nevertheless incorporate the mixing or processing of fluids. For example, the fluid processing system can be used in the production of media, chemicals, food products, beverages, and other liquid products. By way of example and not by limitation, solutions and/or suspensions in the fluid processing system can include one or more biocomponents, including fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components that are grown in fluid processing systems include E. coli, yeast, bacillus, and CHO cells. Solutions and/or suspensions in the fluid processing system can also comprise cell-therapy cultures and cells and microorganisms. Different media compositions known in the art can be used to accommodate the specific cells or microorganisms grown and the desired end product. In some uses, the fluid processing systems primarily grow and recover cells for subsequent use (e.g., preparing vaccine materials from the cells themselves). But in many uses, the ultimate purpose of growing cells in the fluid processing system is to produce and later recover biological products (such as recombinant proteins, viral vectors, etc.) that are exported from the cells into the growth medium. It is also common to use the fluid processing system to grow cells in a master batch to prepare a specific volume, density, concentration, CFU, and/or aliquot of cells for subsequent use as an inoculant for multiple subsequent batches of cells grown to recover biological products.

The disclosed fluid processing systems are designed so that a majority of the system components, including the foam breaker device, that contact the material being processed can be disposed of after each use. As a result, the systems substantially eliminate the burden of cleaning and sterilization required by conventional stainless-steel mixing and processing systems. This feature also ensures that sterility can be consistently maintained during repeated processing of multiple batches.

Various embodiments of a fluid processing system are described below, including fluid processing systems that can include and/or be modified to include one or more foam breaker devices for reducing foam volume and/or foam formation in the fluid processing system.

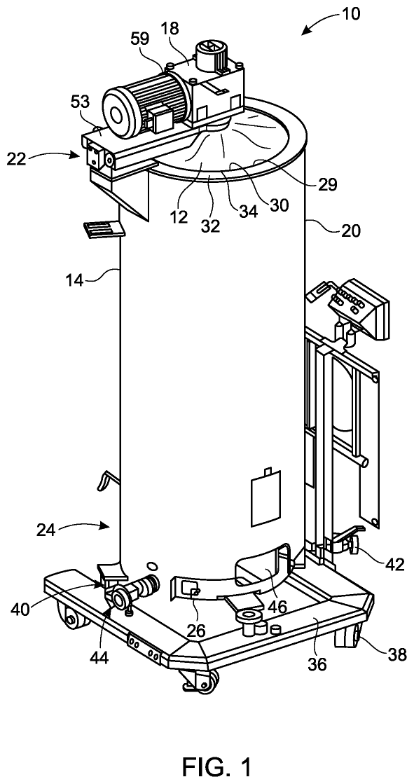

FIG. 1 illustrates an embodiment of a fluid processing system 10 for mixing and processing a variety of fluids. In general, fluid processing system 10 comprises a container 12 that is disposed within a rigid support housing 14. A mixer system 18 is designed for mixing and/or suspending components within container 12. The various components of fluid processing system 10 will now be discussed in greater detail.

With continued reference to FIG. 1, support housing 14 can include a substantially cylindrical sidewall 20 that extends between an upper end 22 and an opposing lower end 24. Lower end 24 has a floor 26 mounted thereto. Support housing 14 has an interior surface 29 that bounds a chamber 30. An annular lip 32 is formed at upper end 22 and bounds an opening 34 to chamber 30. Floor 26 of support housing 14 rests on a cart 36 having wheels 38. Support housing 14 is removably secured to cart 36 by connectors 40. Cart 36 enables selective movement and positioning of support housing 14. In alternative embodiments, however, support housing 14 need not rest on cart 36 but can rest directly on a floor or other structure.

Although support housing 14 is shown as having a substantially cylindrical configuration, in alternative embodiments support housing 14 can have any desired shape capable of at least partially bounding a compartment. For example, sidewall 20 need not be cylindrical but can have a variety of other transverse, cross sectional configurations such as polygonal, elliptical, or irregular. Furthermore, it is appreciated that support housing 14 can be scaled to any desired size. For example, it is envisioned that support housing 14 can be sized so that chamber 30 can hold a volume of less than 50 liters or more than 1,000 liters. Support housing 14 can be made of metal, such as stainless steel, and/or other materials.

In some embodiments, fluid processing system 10 is configured for regulating the temperature of the fluid that is contained within container 12 disposed within support housing 14. By way of example and not by limitation, electrical heating elements can be mounted on or within support housing 14. The heat from the heating elements is transferred either directly or indirectly to container 12. Alternatively, in the depicted embodiment support housing 14 is jacketed with one or more fluid channels being formed therein. The fluid channels have a fluid inlet 42 and a fluid outlet 44 that enables a fluid, such as water or propylene glycol, to be pumped through the fluid channels. By heating, cooling or otherwise controlling the temperature of the fluid that is passed through the fluid channels, the temperature of support housing 14 can be regulated which in turn regulates the temperature of the fluid within container 12 when container 12 is disposed within support housing 14. Other conventional devices can also be used such as by applying gas burners to support housing 14 or pumping the fluid out of container 12, heating or cooling the fluid and then pumping the fluid back into container 12. When using container 12 as part of a bioreactor or fermenter, the means for heating can be used to heat the culture within container 12 to a temperature in a range between about 30° C. to about 40° C. Other temperatures can also be used.

Support housing 14 can have one or more openings 46 formed on the lower end of sidewall 20 and on floor 26 to enable gas and fluid lines to couple with container 12 and to enable various probes and sensors to couple with container 12 when container 12 is within support housing 14. Further disclosure on support housing 14 and alternative designs thereof is disclosed in U.S. Pat. No. 7,682,067 and US Patent Publication No. 2011-0310696, which are incorporated herein by reference.

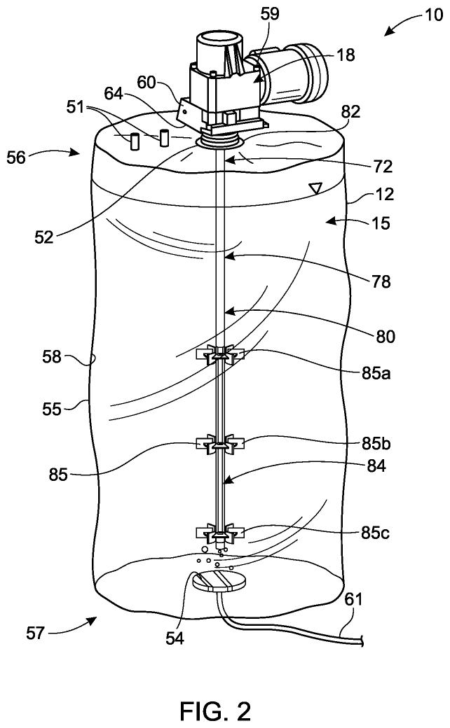

FIG. 2 shows container 12 coupled with mixer system 18. Container 12 has a side 55 that extends from an upper end 56 to an opposing lower end 57. Container 12 also has an interior surface 58 that bounds the inner chamber 15 in which a portion of mixer system 18 is disposed. In the embodiment depicted, container 12 comprises a flexible bag. Formed on container 12 are a plurality of ports 51 that communicate with inner chamber 15. Although only two ports 51 are shown, it is appreciated that container 12 can be formed with any desired number of ports 51 and that ports 51 can be formed at any desired location on container 12 such as upper end 56, lower end 57, and/or alongside 55. Ports 51 can be the same configuration or different configurations and can be used for a variety of different purposes. For example, ports 51 can be coupled with fluid lines for delivering media, cell cultures, and/or other components into and/or out of container 12.

Ports 51 can also be used for coupling probes to container 12. For example, when container 12 is used as a bioreactor for growing cells or microorganisms, ports 51 can be used for coupling probes such as temperatures probes, pH probes, dissolved oxygen probes, and the like. Examples of ports 51 and how various probes and lines can be coupled thereto is disclosed in United States Patent Publication No. 2006-0270036, published Nov. 30, 2006 and United States Patent Publication No. 2006-0240546, published Oct. 26, 2006, which are incorporated herein by specific reference. Ports 51 can include various tubing and can be used for coupling container 12 to secondary containers and to other desired fittings.

In various embodiments of the present invention, means are provided for delivering a gas into the lower end of container 12. By way of example and not by limitation, as also depicted in FIG. 2, a sparger 54 can be either positioned on or mounted to lower end 57 of container 12 for delivering a gas to the fluid within container 12. As is understood by those skilled in the art, various gases are typically required in the growth of cells or microorganisms within container 12. The gas typically comprises air that is selectively combined with oxygen, carbon dioxide and/or nitrogen. However, other gases can also be used. The addition of these gases can be used to regulate the dissolved oxygen and CO₂ content and to regulate the pH of a culture solution. Depending on the application, sparging with gas can also have other applications. A gas line 61 is coupled with sparger 54 for delivering the desired gas to sparger 54. Gas line 61 need not pass through lower end 57 of container 12 but can extend down from upper end 56 or from other locations.

Sparger 54 can have a variety of different configurations. For example, sparger 54 can comprise a permeable membrane or a fritted structure comprised of metal, plastic or other materials that dispense the gas in small bubbles into container 12. Smaller bubbles can permit better absorption of the gas into the fluid. In other embodiments, sparger 54 can simply comprise a tube, port, or other type of opening formed on or coupled with container 12 through which gas is passed into container 12. In contrast to being disposed on container 12, the sparger can also be formed on or coupled with mixer system 18. Examples of spargers and how they can be used in the present disclosure are disclosed in United States Patent Publication Nos. 2006-0270036 and 2006-0240546 which are incorporated by reference. Other conventional spargers can also be used. It is appreciated that in some embodiments and uses that a sparger may not be required.

In the depicted embodiment, container 12 has an opening 52 that is sealed to a rotational assembly 82 of mixer system 18, which will be discussed below in greater detail. As a result, the inner chamber 15 is sealed closed so that it can be sterilized and used in processing sterile fluids. During use, container 12 is disposed within chamber 30 of support housing 14 as depicted in FIG. 1. Container 12 is supported by support housing 14 during use and can subsequently be disposed of following use. In one embodiment, container 12 is comprised of a flexible, water impermeable material such as a low-density polyethylene or other polymeric sheets or film having a thickness in a range between about 0.1 mm to about 5 mm with about 0.2 mm to about 2 mm being more common. Other thicknesses can also be used. The material can be comprised of a single ply material or can comprise two or more layers which are either sealed together or separated to form a double wall container. Where the layers are sealed together, the material can comprise a laminated or extruded material. The laminated material comprises two or more separately formed layers that are subsequently secured together by an adhesive.

The extruded material comprises a single integral sheet that comprises two or more layers of different materials that can be separated by a contact layer. All of the layers are simultaneously co-extruded. One example of an extruded material that can be used in the present invention is the Thermo Scientific CX3-9 film available from Thermo Fisher Scientific. The Thermo Scientific CX3-9 film is a three-layer, 9 mil cast film produced in a cGMP facility. The outer layer is a polyester elastomer coextruded with an ultra-low density polyethylene product contact layer. Another example of an extruded material that can be used in the present invention is the Thermo Scientific CX5-14 cast film also available from Thermo Fisher Scientific. The Thermo Scientific CX5-14 cast film comprises a polyester elastomer outer layer, an ultra-low density polyethylene contact layer, and a barrier layer disposed therebetween.

The material is approved for direct contact with living cells and is capable of maintaining a sterile solution. In such an embodiment, the material can also be sterilizable such as by radiation. Examples of materials that can be used in different situations are disclosed in U.S. Pat. No. 6,083,587 which issued on Jul. 4, 2000 and United States Patent Publication No. US 2003-0077466 A1, published Apr. 24, 2003, which are hereby incorporated by specific reference.

In one embodiment, container 12 comprises a two-dimensional pillow style bag wherein two sheets of material are placed in overlapping relation and the two sheets are bounded together at their peripheries to form the internal compartment. Alternatively, a single sheet of material can be folded over and seamed around the periphery to form the internal compartment. In another embodiment, the containers can be formed from a continuous tubular extrusion of polymeric material that is cut to length and is seamed closed at the ends.

In still other embodiments, container 12 can comprise a three-dimensional bag that not only has an annular side wall but also a two-dimensional top-end wall and a two-dimensional bottom-end wall. Three-dimensional containers comprise a plurality of discrete panels, typically three or more, and more commonly four or six. Each panel is substantially identical and comprises a portion of the side wall, top-end wall, and bottom-end wall of the container. The corresponding perimeter edges of each panel are seamed together. The seams are typically formed using methods known in the art such as heat energies, radiofrequency (RF) energies, sonics, or other sealing energies.

In alternative embodiments, the panels can be formed in a variety of different patterns. Further disclosure with regard to one method of manufacturing three-dimensional bags is disclosed in United States Patent Publication No. US 2002-0131654 A1, published Sep. 19, 2002, which is hereby incorporated by reference.

It is appreciated that container 12 can be manufactured to have virtually any desired size, shape, and configuration. For example, container 12 can be formed having a compartment sized to 10 liters, 30 liters, 100 liters, 250 liters, 500 liters, 750 liters, 1,000 liters, 1,500 liters, 3,000 liters, 5,000 liters, 10,000 liters or other desired volumes. The size of the compartment can also be in the range between any two of the above volumes. Although container 12 can be any shape, in one embodiment container 12 is specifically configured to be complementary or substantially complementary to chamber 30 of support housing 14. It is desirable that when container 12 is received within chamber 30, container 12 is at least generally uniformly supported by support housing 14. Having at least general uniform support of container 12 by support housing 14 helps to preclude failure of container 12 by hydraulic forces applied to container 12 when filled with fluid.

Although in the above discussed embodiment container 12 has a flexible, bag-like configuration, in alternative embodiments it is appreciated that container 12 can comprise any form of collapsible container or semi-rigid container. Container 12 can also be transparent or opaque and can have ultraviolet light inhibitors incorporated therein.

Mixer system 18 is used for mixing and/or suspending a culture or other solution or suspension within container 12. As depicted in FIG. 2, mixer system 18 generally comprises a drive motor assembly 59 that is mounted on support housing 14 (FIG. 1), an impeller assembly 78 coupled to and projecting into container 12, and a drive shaft 72 that extends between drive motor assembly 59 and impeller assembly 78. In some embodiments, the drive shaft 72 can include more than one part coupled together and caused to rotate by the drive motor assembly 59. For example, impeller assembly 78 can be a part of and/or coupled to the drive shaft 72 such that when the drive shaft 72 rotates, the impeller assembly 78, including mounted impellers 85 and/or foam breaker devices, rotates. As such, as disclosed herein, when reference is made to a feature being coupled to the drive shaft 72, such feature can be coupled directly or indirectly to the drive shaft 72.

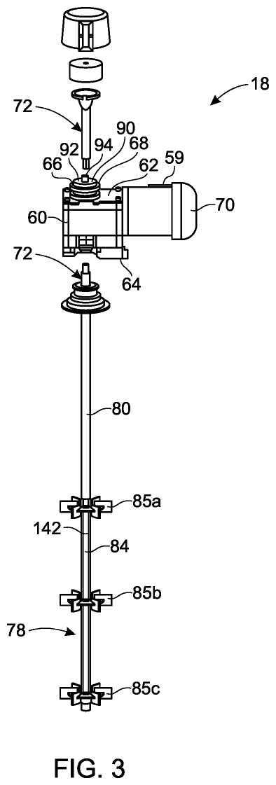

Turning to FIG. 3, drive motor assembly 59 comprises a housing 60 having a top surface 62 and an opposing bottom surface 64 with an opening 66 extending through housing 60 between surfaces 62 and 64. A tubular motor mount 68 is rotatably secured within opening 66 of housing 60 and bounds a passage 90 extending therethrough. As depicted in FIG. 3, the upper end of motor mount 68 terminates at an ends face 92 having a locking pin 94 outwardly projecting therefrom. A thread can encircle motor mount 68 adjacent to end face 92. A drive motor 70 is mounted to housing 60 and engages with motor mount 68 so as to facilitate select rotation of motor mount 68 relative to housing 60. As depicted in FIG. 1, drive motor assembly 59 is coupled with support housing 14 by a bracket 53. In alternative embodiments, however, drive motor assembly 59 can be mounted on a separate structure adjacent to support housing 14.

Drive shaft 72 is configured to pass through motor mount 68 and thus through housing 60. Further disclosure with regard to the mixer system including the drive shaft is disclosed in U.S. Pat. No. 10,335,751, granted Jul. 2, 2019, which is hereby incorporated by reference.

For example, the drive shaft 72 can include a variety of features, such as include a flexible drive shaft portion, a rigid drive shaft portion, a foldable drive shaft portion, and/or a ladder drive shaft portion that can form straight and/or helical configurations. As will be described in greater detail below, the drive shaft 72 can cause at least one impeller and at least one foam breaker device to rotate along with rotation of the drive shaft 72.

As depicted in FIG. 3, the drive shaft 72 can be coupled to a first tubular connector 80, and the first tubular connector 80 can be coupled to a second tubular connector 84. The first tubular connector 80 and/or the second tubular connector 84 can have a transverse cross-section that is polygonal, elliptical, or some other non-circular configuration. For example, the transverse cross-section can be polygonal having five, six, seven, or more sides.

Exterior surface 142 of second tubular connector 84 is configured to receive one or more impellers 85, such as impellers 85a-85c, so that they can be fixed thereon. As used in the specification and appended claims, the term “impeller” is broadly intended to include all conventional types of impellers and impeller blades along with other structures that can be mounted on second tubular connector 84 so that when second tubular connector 84 is rotated within container 12, the structures can uniformly mix the fluid within container 12. Further disclosure with regard to impellers 85 is disclosed in U.S. Pat. No. 10,335,751, granted Jul. 2, 2019, which is hereby incorporated by reference.

As discussed above, mixing of liquids in the container 12 (e.g., bioreactor) can cause foam to form, such as above a liquid in the container 12. The foam can cause damage to filters and processing of the liquid and/or biological material. The present disclosure includes a variety of foam breaker devices 200 that are configured to disrupt the foam such that it returns to liquid form. In some embodiments, the foam breaker device can be adaptable to current mixing systems, such as configured to be coupled to a variety of drive shafts. The foam breaker devices can be provided in a variety of sizes (e.g., diameters) and shapes, such as a cone shape, for being positioned within a variety of fluid pathways and chambers.

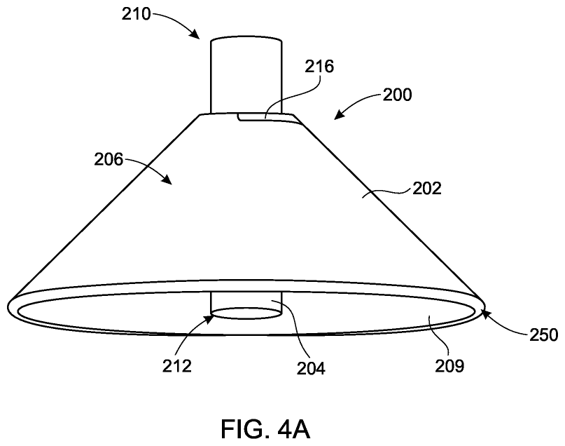

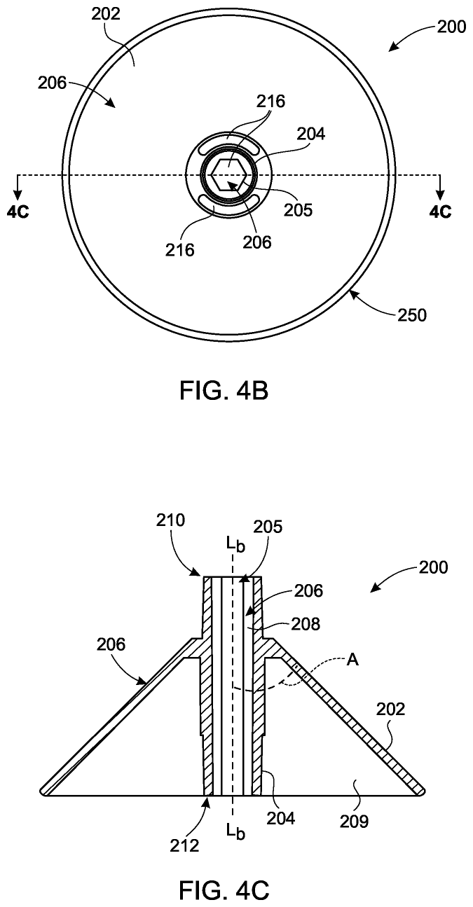

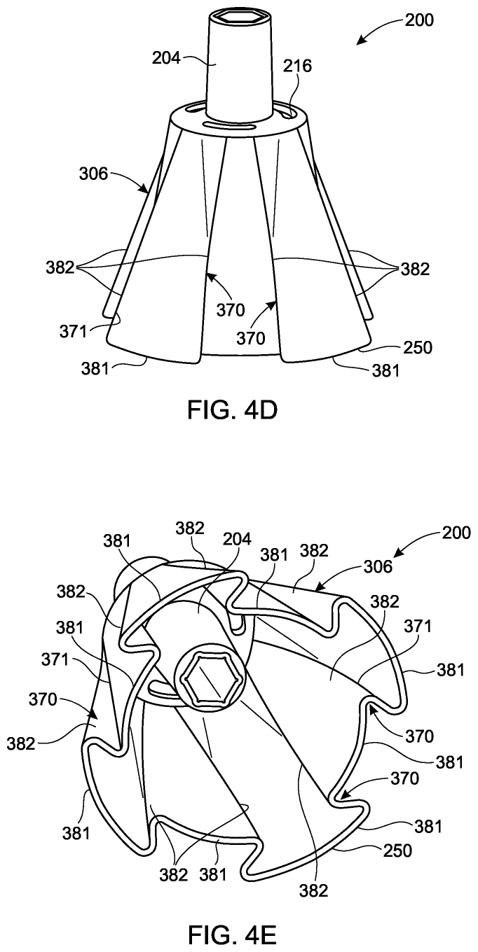

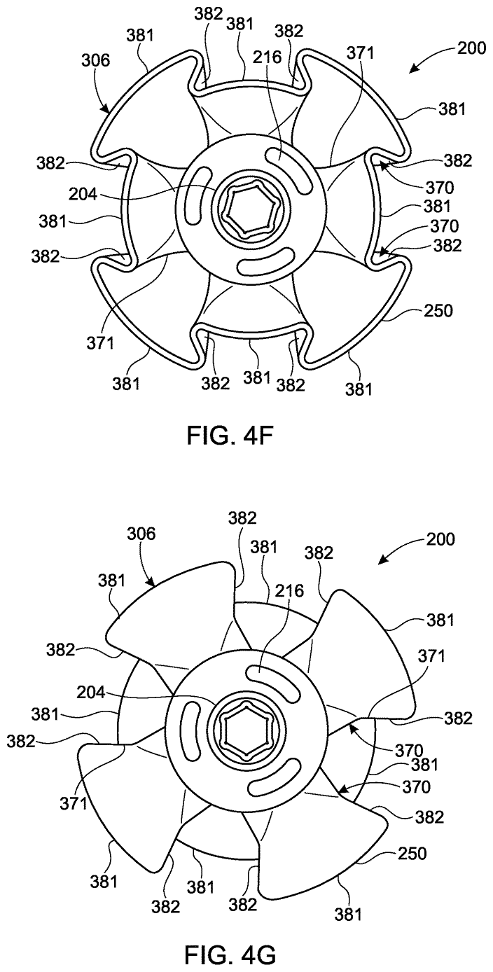

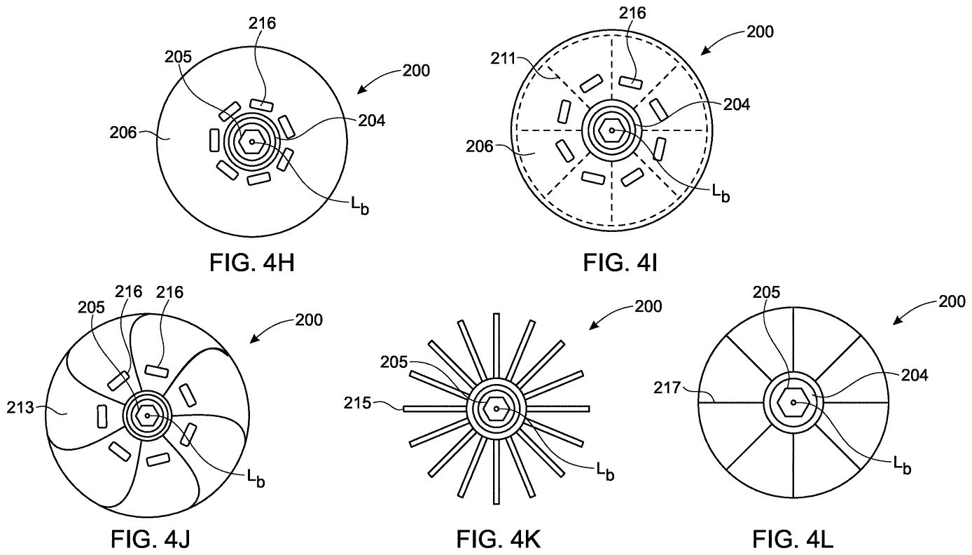

FIGS. 4A-4G illustrate embodiments of a foam breaker device 200 that can be coupled directly or indirectly to a part of an embodiment of the drive shaft 72 (FIG. 2) of the fluid processing system 10 (FIG. 1). For example, the foam breaker device 200 can be coupled to the second tubular connector 84 (FIG. 2) that can be coupled directly or indirectly to the drive shaft 72. In other words, foam breaker device 200 can be configured to be indirectly or directly coupled to drive motor assembly 59 and in turn, be controlled by drive motor assembly 59. The foam breaker 200 can be coupled directly or indirectly to the drive shaft 72 such that when the drive shaft 72 rotates, the foam breaker device 200 is caused to rotate along a first axis also known as a longitudinal axis or L_{b} of the foam breaker device 200, as shown in FIG. 4C.

As shown in FIG. 4A, the foam breaker device 200 can have a body 202 including a mounting hub 204 extending along the longitudinal axis L_{b} of the foam breaker device 200. The mounting hub 204 can include a coupling feature 206, as shown in FIG. 4B, for directly or indirectly coupling the foam breaker device 200 to the drive shaft 72. For example, the coupling feature 206 can include a passageway 205 that extends through the mounting hub 204 and along the longitudinal axis L_{b}. The mounting hub 204 can include a proximal end 210 and a distal end 212 and the passageway 205 can extend between the proximal end 210 and the distal end 212 of the mounting hub 204, such as extend along the entire length of the mounting hub 204. The passageway 205 can include an inner passageway diameter that is sized to allow the mounting hub 204 to couple to the second tubular connector 84. For example, the passageway 205 can be sized and shaped to allow inner passageway walls 208 of the passageway 205, as shown in FIG. 4C, to have a translational sliding engagement with an outer shaft surface of the drive shaft 72 and/or a part coupled directly or indirectly with the drive shaft 72 (e.g., the second tubular connector 84). For example, such translational sliding engagement can allow the foam breaker device 200 to slidably translate along the second tubular connector 84, such as to position and secure the foam breaker device 200 along the second tubular connector 84.

In some embodiments, the passageway 205 can be configured to be rotationally fixed relative to the drive shaft 72. For example, the foam breaker device 200 can be prevented from rotating independent from or relative to the drive shaft 72 or the second tubular connector 84. As shown in FIG. 4B, the passageway 205 can include a hexagonal shaped cross-section that is approximately the same as or similar to an embodiment of the second tubular connector 84 having a hexagonal shaped cross-section. Such configuration can prevent at least independent rotation of the foam breaker device 200 relative to the second tubular connector 84 and drive shaft 72. Furthermore, such engagement causes rotation of the foam breaker device 200 along its longitudinal axis L_{b} as a result of rotation of the drive shaft 72. Other coupling features 206, including various shaped passageways 205 (e.g., square, hexagon, etc.), configured to couple the foam breaker device 200 directly and/or indirectly to the drive shaft 72 and/or second tubular connector 84 are within the scope of this disclosure.

As shown in FIGS. 4A-4C, the foam breaker device 200 can include a wall structure 206 that extends circumferentially from the mounting hub 204 at an angle A relative to the longitudinal axis L_{b} of the foam breaker device 200. In this case, the wall structure 206 has a cone shaped wall. The cone shaped wall 206 can reduce a foam volume and/or reduce foam formation as a result of at least a part of the cone shaped wall 206 rotating within the foam volume. For example, rotation of the cone shaped wall 206 can cause a shearing effect to the foam which destructs the structure of the foam. This can cause the foam to turn into a liquid, thereby reducing the foam volume as some of the foam in the foam volume is turned into liquid foam. The cone shaped wall 206 can include an outer surface 207 and an inner surface 209, as shown in FIG. 4C. The outer surface 207 and/or the inner surface 209 can be substantially planar, as shown in FIG. 4C.

The cone shaped wall 206 can extend from the mounting hub 204 at an angle A relative to the longitudinal axis L_{b} of the foam breaker device 200, as shown in FIG. 4C. In some embodiments, the cone shaped wall 206 can extend relative to the longitudinal axis L_{b} of the foam breaker device 200 at an angle A that is within an angle range of approximately 10 degrees to approximately 90 degrees, such as 30 degrees to approximately 50 degrees. In some embodiments, the angle A can be less than 10 degrees or more than 80 degrees. At least the cone shaped wall 206 can be formed out of a hydrophobic material. In some embodiments, the cone shaped wall 206 can be formed out of a hydrophilic material. In some embodiments, the cone shaped wall 206 can be formed out of a material that is flexible and/or foldable.

As shown in FIG. 4B, the cone shaped wall 206 of the foam breaker device 200 includes a perimeter 250 that extends along a distal end of the cone shaped wall 206. For example, the perimeter 250 can have a circular shape, as shown in FIG. 4B, and/or can have a perimeter that changes size and/or shape. The perimeter 250 can include any of a variety of sizes and shapes without departing from the scope of this disclosure.

In some embodiments, the foam breaker device 200 can include a cone shaped wall 206 that is flexible and/or foldable, which can allow the foam breaker device 200 to form a compact configuration and fit through existing openings in a variety of fluid processing systems 10 (e.g., port 51 at FIG. 2), such as openings that are smaller in diameter compared to a diameter of the perimeter 250 of the foam breaker device 200 when not in a collapsed configuration (e.g., an extended configuration, as shown in FIG. 4A). As such, the foam breaker device 200 that is collapsible or foldable can be coupled to a variety of existing fluid processing systems 10, such as by passing the foam breaker device 200 in a collapsed configuration through an existing opening in a fluid processing system 10 to modify the fluid processing system 10 to include at least one foam breaker device 200.

FIGS. 4D-4G illustrate an embodiment of the foam breaker device 200 including a collapsible cone shaped wall 306. For example, the collapsible cone shaped wall 306 can be formed out of a foldable and/or flexible material that allows the foam breaker device 200 to transition between an extended configuration (e.g., the foam breaker device shown in FIG. 4A) and a collapsed configuration, as shown in FIG. 4E. The foam breaker device 200 can transition from the extended configuration into the collapsed configuration by folding the collapsible cone shaped wall 306, such as by forming one or more folds and/or pleats 370 along the collapsible cone shaped wall 306.

For example, an outer diameter of the collapsible cone shaped wall 306 in the collapsed configuration can be smaller than an outer diameter of the collapsible cone shaped wall 306 in the extended configuration. This can allow the foam breaker device 200 in the collapsed configuration to pass through smaller openings and modify a variety of fluid processing systems 10 (e.g., couple to existing drive shafts 72, such as in FIG. 2).

As shown in FIGS. 4E and 4F, the foam breaker device 200 can form one or more pleats 370 along a length of the collapsible cone shaped wall 306, which can allow the foam breaker device 200 to form the collapsed configuration. For example, the foam breaker device 200 can include eight pleats 370, as shown in FIG. 4E, however, the foam breaker device 200 can include more or less than eight pleats 370 without departing from the scope of this disclosure. Each pleat 370 can have a same or similar configuration or can be different, such as including folds in a same and/or opposing direction.

In some embodiments, one or more pleats 370 can be pre-formed into the collapsible cone shaped wall 306, such as to form a predefined collapsed configuration. In some embodiments, the collapsible cone shaped wall 306 is allowed to form one or more pleats 370 along any number of a variety of locations along the collapsible cone shaped wall 306.

In some embodiments, the collapsible cone shaped wall 306 can be made out of one or more of a rigid, flexible, hydrophobic, hydrophilic, and shape-memory material. For example, as shown in FIGS. 4D-4G, the cone shaped wall 306 can include rigid sections 381 at least partly separated by flexible sections 382 that allow the collapsible cone shaped wall 306 to achieve the collapsed and extended configurations. For example, the rigid sections 381 can be made out of a same or different material than the flexible sections 382, however, the rigid sections 381 can have a material property that is more rigid compared to the flexible sections 382. As shown in FIGS. 4E and 4F, the flexible sections 382 can allow at least one pleat 370 to form therealong, such as due to the flexible section 382 being formed of a flexible material. The rigid sections 381 and flexible sections 382 can extend along the length of the collapsible cone shaped wall 306, such as a length extending between the mounting hub 204 and the perimeter 250, including the entire length between the mounting hub 204 and the perimeter 250 of the cone shaped wall 306. The flexible sections 382 can allow the rigid sections 381 to pivot towards a longitudinal axis of the foam breaker device 200 to form the collapsed configuration, as well as pivot away from the longitudinal axis to form the extended configuration.

For example, as shown in FIGS. 4E and 4F, the ability of the flexible sections 382 to fold and/or form pleats 370 can allow the rigid sections 382 to at least partially stack and/or overlap with at least one adjacent rigid section 382. This can allow the collapsible cone shaped wall 306 to form compact collapsed configurations. Such compact collapsed configurations can allow the foam breaker device 200 to fit through smaller openings, as well as assist in improved packaging (e.g., smaller packaging) of the foam breaker device 200. Additionally, the compact collapsed configurations can allow the foam breaker device 200 to achieve improved assembly and incorporation into a bioproduction vessel (e.g., due to fitting through smaller ports and/or passageways) and/or in a component thereof (e.g., filter). Any number of rigid sections 381 and flexible sections 382 can be included in an embodiment of the collapsible cone shaped wall 306. For example, as shown in FIGS. 4E and 4F, the collapsible cone shaped wall 306 can include eight rigid sections 381 separated by eight flexible sections 382.

In some embodiments, the collapsible cone shaped wall 306 is configured to form a collapsed configuration when the foam breaker device 200 is not rotating and configured to form the extended configuration when the foam breaker device 200 is rotating. As shown in FIGS. 4D-4G, each pleat 370 can form and extend along a length of the collapsible cone shaped wall 306, such as a length extending between the mounting hub 204 and the perimeter 250, including the entire length between the mounting hub 204 and the perimeter 250.

As shown in FIGS. 4A and 4G, the cone shaped wall 206 of the foam breaker device 200 can include a through hole 216 positioned adjacent the mounting hub 204 for allowing one or more of a fluid, gas, and air to pass through the through hole 216. In some embodiments, the foam breaker device 200 can include more than one through hole 216 that can each extend circumferentially around the longitudinal axis L_{b} and adjacent the mounting hub 204. Other shapes and configurations of the through hole 216 are within the scope of this disclosure. For example, the through hole 216 can provide a passageway for foam that has formed into liquid to travel through the foam breaker device 200 (e.g., via the through hole 216) and flow into liquid contained below the foam. The foam breaker device 200 including the mounting hub 204 and wall structure 206 can be printed as one integral unit. Optionally the wall structure 206 can be attached to the mounting hub 204 by adhesive techniques. Other attachment techniques fall under the scope of this disclosure.

FIGS. 4H-4L illustrate various other embodiments of the foam breaker device 200. For example, a flat disc-shaped wall structure 206 can extend circumferentially around the mounting hub 204 and include symmetrically disposed apertures 216 around the mounting hub 204 as shown in FIG. 4H. Further, the wall structure 206 can include a memory metallic wire structure 211 (e.g., a memory metal alloy such as nitinol or Nickel titanium including Nitinol 55, and Nitinol 60) embedded in the wall structure 206 as shown in FIG. 4I. In other words, memory metallic wire substantially supports the wall structure 206 of the foam breaker device 200 while in use. The memory metallic wire 211 structure can provide for extending the wall structure 206 at an angle over a range of approximately 10 degrees to approximately 90 degrees relative to the longitudinal axis L_{b} of the foam breaker device 200 owing to its memory properties. Further, wall structure 206 can include micro texturing or macro texturing on both the inside and outside surfaces. Such micro or macro texturing can help in quick foam breaking owing to the surface properties of the textures. In another example, partially overlapping fin structures 213 can extend radially outward from the mounting hub 204 as shown in FIG. 4J. Each of the fin structures can include apertures 216 proximate to the mounting hub 204. Further, the fin structures 213 can be arranged in a clockwise or anti-clockwise direction of overlapping fin structures. In yet another example, the wall structure can be formed by a plurality of adjacently placed bristles or tubular structures 215 extending radially outward from the mounting hub 204 as shown in FIG. 4K. In yet another example, a metallic wire frame 217 can extend circumferentially from the mounting hub 204. An eight-part wire frame structure is shown in FIG. 4L, but other frame structures including more or less than eight-part frame structures fall under the scope of the disclosure. In common, mounting hub 204 shown in the various embodiments in FIGS. 4H-4L can include a coupling feature 205 extending along a longitudinal axis L_{b} for coupling directly or indirectly to drive shaft 72 or drive motor assembly 59 as mentioned above.

One or more foam breaker devices 200 can be included in an embodiment of a fluid processing system 10, such as more than one foam breaker device 200 coupled to a drive shaft 72 extending along an inner chamber 15 of a container 12 of the processing system 10. The container 12 can include a variety of shapes, sizes and characteristics. For example, the container 12 can be formed of a rigid, semi-rigid or flexible material. In some embodiments, the container 12 is one or more of a bioreactor, a flexible and disposable bag, a condenser, a fermenter, and a mixing chamber of the fluid processing system 10.

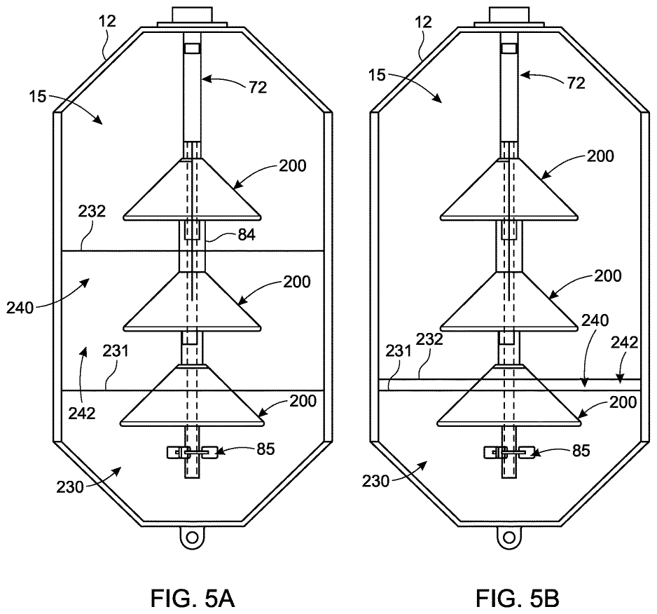

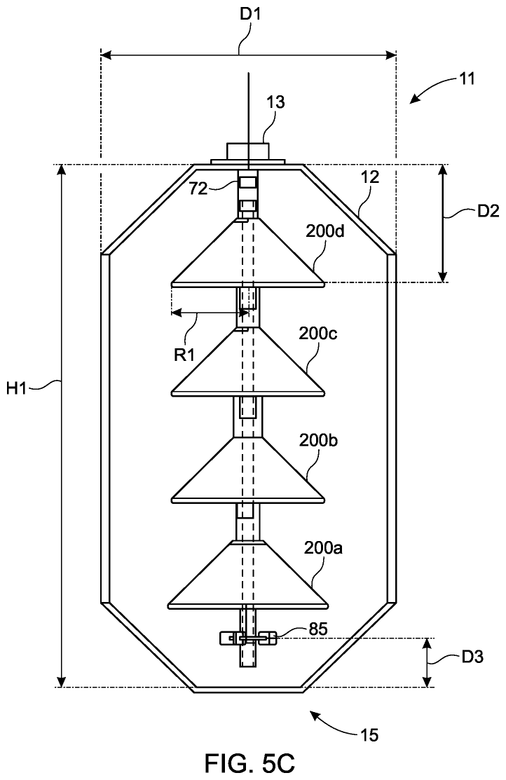

FIGS. 5A and 5B illustrate an embodiment of the drive shaft 72 including three foam breaker devices 200 coupled to a part of the drive shaft 72, such as along the second tubular connector 84. For example, one or more impellers 85 can also be coupled along the same drive shaft 72 and/or second tubular connector 84, as shown in FIGS. 5A and 5B. As such, during rotation of the drive shaft 72, a liquid 230 contained in the inner chamber 15 can be mixed as a result of the one or more impellers 85 rotating, and a foam 240 formed in the inner chamber 15 can be reduced as a result of rotation of at least one foam breaker device 200.

As shown in FIG. 5A at least one impeller 85 can be positioned in the liquid 230 and at least one foam breaker device 200 can be positioned in the foam 240. As a result of rotation of the drive shaft 72 (e.g., due to activation of the mixer system 18) the at least one impeller 85 can rotate and mix the liquid 230 and at least one foam breaker device 200 can rotate and reduce foam 240 in the inner chamber 15. For example, FIG. 5B illustrates a reduction in foam volume 242 compared to the foam volume 242 present in the inner chamber 15 prior to rotation of the foam breaker devices 200 (as shown in FIG. 5A). In some embodiments, at least one foam breaker device 200 is positioned above the liquid surface. When submerged in the liquid 230, the impeller 85 can stabilize the drive shaft 72, such as during rotation of the drive shaft 72 and/or impeller 85.

As shown in FIGS. 5A and 5B, the foam volume 242 can be determined based on a thickness of the foam and dimensions of the inner chamber 15 (e.g., diameter). The thickness of the foam 240 can extend between a top surface 231 of the liquid 230 and a top surface 232 of the foam 240. For example, the thickness of the foam 240 (e.g., distance between top surface 231 of liquid 230 and top surface 232 of foam 240) is smaller in FIG. 5B (e.g., after rotation of the foam breaker devices 200) compared to the thickness of foam 240 in FIG. 5A (e.g., prior to rotation of the foam breaker devices 200). As such, rotation of at least one foam breaker device 200 in foam 240 can reduce a foam volume 242 and/or thickness of the foam 240. During operation, impeller 85 is ideally submerged under the top surface 231 of the liquid 230 where it can stabilize the drive shaft 72 while rotating.

In some embodiments, the foam breaker device 200 rotates at a speed of approximately 50 revolutions per minute (RPMs) to 1200 RPMs. Other rotational speeds are within the scope of this disclosure (e.g., greater than 1200 RPM, such as at least 2000 RPM) that allow the foam breaker device 200 to cause disruption to foam thereby causing the foam to form into a liquid. In some embodiments, the foam breaker device 200 includes an edge speed (edge speed is a distance traveled by any selected point on a peripheral edge of the foam breaker device 200 in a set time) ranging between 90.0 inches/second to 255 inches/second. In an example operation process of foam breaker device, 200, a rotation of the foam breaker device 200 can be initiated at medium edge speed (140-160 inches/second) to effectuate foam breaking. If the foam reduction is not effective in 3-5 seconds, the rotation can be ramped up to a fast edge speed (240-255 inches/second) to effectuate foam reduction. Hence, foam breaker device 200 provides for effectively reducing foam in a short interval of time.

In some embodiments, the perimeter 250 of the foam breaker device 200 in an extended configuration (FIG. 4A) can include a diameter that is sized such that the diameter is greater than half of an inner diameter of the inner chamber 15 of the container 12. For example, the perimeter 250 of the cone shaped wall 206 can include a diameter that is greater than three-quarters of an inner diameter of the inner chamber 15 of the container 12. In some embodiments, the diameter can be approximately 4 inches to approximately 10 inches. Other dimensions are within the scope of this disclosure. For example, the 250 of the cone shaped wall 206 (FIG. 4A) can have a diameter that is approximately the same as an inner diameter of the inner chamber 15 of the container 12. In some embodiments, the ratio of a diameter of the foam breaker device to an inner diameter of the inner chamber of the container can be 1:2. Other ratios of the diameter of the foam breaker device to the inner diameter of the inner chamber of the container are within the scope of this disclosure, including 1:1.5, 1:3, 1:4, etc.

In some embodiments, the diameter of the perimeter 250 is approximately the same as a diameter of a port 51 (FIG. 2) such that the foam breaker device 200 can have a sliding fit with a port 51. Foam breaker devices 200 having larger perimeters 250 can be rotated at a slower rotational speed (e.g., RPM) compared to foam breaker devices 200 having a smaller perimeter 250 to achieve a similar result (e.g., foam dispersion, foam prevention). This can be due to the greater speed of rotation experienced along the larger perimeter 250 cone shaped walls 206 compared to speeds of rotation experienced along the smaller perimeter 250 cone shaped walls 206 when rotating at a same speed.

FIG. 5C illustrates an embodiment of the drive shaft 72 including four foam breaker devices 200a-d coupled to a part of the drive shaft 72, but any number of foam breaker devices, more or less than four foam breaker devices can be coupled to a part of the drive shaft 72. For example, container 12 can be a 77.0 L capacity condenser surge foam breaker chamber including the following dimensions: Volume: 77.84 L (2.749 sqft); height H1: 42.0″ (106 cm); Inner Diameter D1: 12.0″ (76.2 cm). Additionally, container 12 can be partly jacketed and partly unjacketed to improve condensation and foam-breaking performance. Each of foam breaker devices 200a-d has a radius R1: 8.0 inches, the foam breaker device 200d proximate to a top end 11 of container 12 being a distance D2: 9.5 inches below a bearing port 13, and an impeller 85 (e.g., Rushton Impeller) being a distance D3: 5.0 inches above a bottom 15 of the container 12 and 3.5 inches below the foam breaker device 200a proximate to the bottom of the container 12. Above embodiment of container 12 is configured to provide support to sustain higher pressures during fluid processing in the container 12. Other dimensions for container 12, including volumes ranging between 1 L to several 100 L, and with varied spacing between foam breaker devices 200a-d to disrupt/break/disintegrate foam are within the scope of this disclosure.

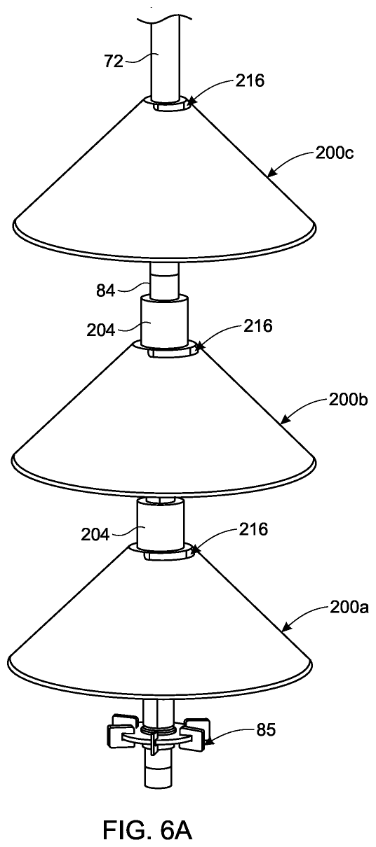

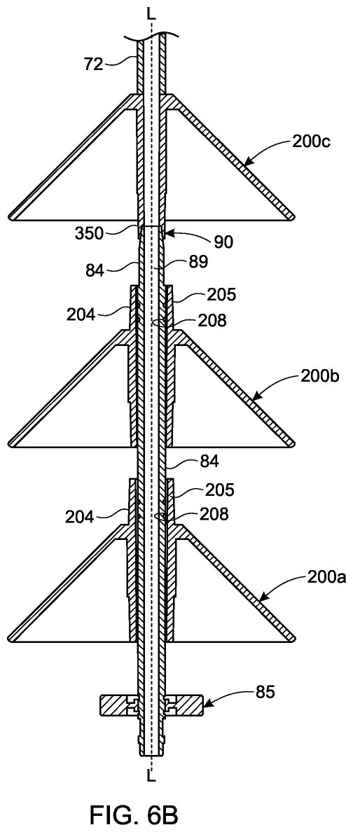

FIGS. 6A and 6B illustrate an embodiment of the relative positioning and coupling of the three foam breaker devices 200a, 200b, 200c and an impeller 85 along the second tubular connector 84. For example, the impeller 85 can be positioned at a most distal position along the second tubular connector 84 and the three foam breaker devices 200a, 200b, 200c can be spaced apart along more proximal positions along the second tubular connector 84. As shown in FIG. 6B, the three foam breaker devices 200a, 200b, 200c can be coupled and aligned to the second tubular connector 84 such that the longitudinal axis of each foam breaker device 200 is coaxial with a longitudinal axis L of the second tubular connector 84.

Each foam breaker device 200a, 200b, 200c can be positioned along the drive shaft 72 and/or second tubular connector 84 in more than one orientation. For example, each foam breaker device 200a, 200b, 200c can form a first orientation with the drive shaft 72 and/or second tubular connector 84 such that the inner surface 209 of the cone shaped wall 206 is directed toward a distal end of the second tubular connector 84 and/or toward the lower end 57 of the container 12, such as shown in FIG. 5A. Additionally, the foam breaker device 200 can form a second orientation with the drive shaft 72 and/or second tubular connector 84 such that the inner surface 209 of the cone shaped wall 206 is directed toward a proximal end of the second tubular connector 84 and/or toward an upper end 56 of the container 12. For example, the passageway 205 can allow the second tubular connector 84 to enter the passageway 205 via the proximal end 210 and/or the distal end 212 of the mounting hub 204 thereby allowing the foam breaker device 200 to form the first or second orientation. The ability to couple the foam breaker device 200 to the drive shaft 72 and/or the second tubular connector 84 provides an efficient way to manufacture and/or modify fluid processing systems to include one or more foam breaker device 200.

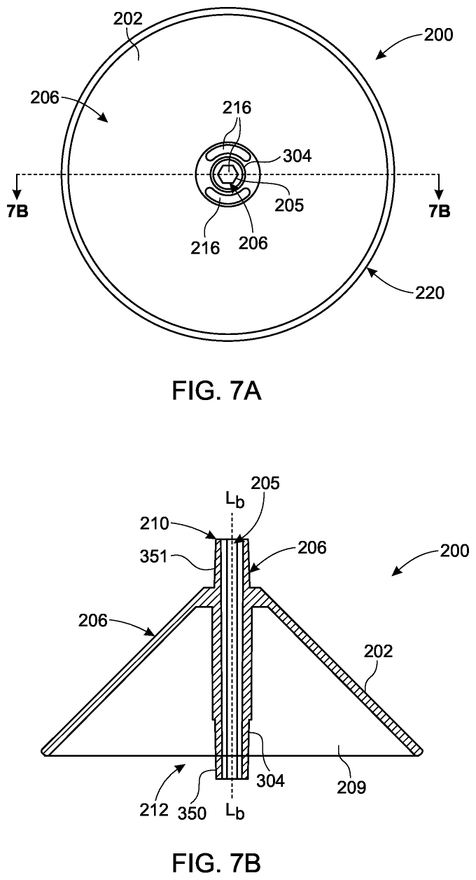

FIGS. 7A and 7B illustrate the foam breaker device 200 that includes another embodiment of the mounting hub 304 including a distal connecting feature 350 and a proximal connecting feature 351. For example, the distal connecting feature 350 can allow the foam breaker device 200 to couple to an end of a part of the drive shaft 72, such as a proximal end of the second tubular connector 84 of the impeller assembly 78, as shown in FIGS. 5A-6B. As such, the proximal connecting feature 351 (FIG. 7B) can couple to a distal end of a part of the drive shaft 72, such as a distal end of the first tubular connector 80 of the impeller assembly 78 (FIG. 3), thereby connecting the second tubular connector 84 to the first tubular connector 80 via the mounting hub 304 (FIG. 7A) of foam breaker device 200. As shown in FIG. 6B, the foam breaker device 200 can couple to an outer surface and/or end of the second tubular connector 84.

The foam breaker device 200 can be adapted to an existing mixing system 18 of a fluid processing system 10 (FIG. 1). For example, a part of the drive shaft 72 and/or a part of the impeller assembly 78 (FIG. 3) can be modified to include one or more foam breaker devices 200. Such modification of a mixing system 18 (FIG. 3) can include removing one or more impellers 85, such as by sliding one or more impellers off of the second tubular connector 84. One or more foam breaker device 200 can be coupled to the second tubular connector 84, as will be described in detail below.

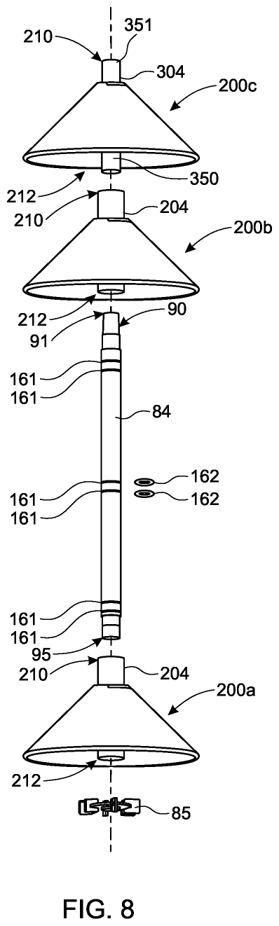

FIG. 8 illustrates three foam breaker devices 200, such as a first foam breaker device 200a, a second foam breaker device 200b, and a third foam breaker device 200c that can be coupled to the second tubular connector 84 of the impeller assembly 78 that couples to the drive shaft 72 (FIG. 2). For example, the first foam breaker device 200a can insert a distal end 95 of the second tubular connector 84 into the proximal end 210 of the passageway 205 of the first foam breaker device 200a and advance the second tubular connector 84 along the passageway 205. As shown in FIG. 8, the second tubular connector 84 can include annular grooves 161 along an outer wall that allows a retainer 162 (e.g., O-ring) to be positioned in each annular groove 161. For example, the first foam breaker device 200a can be positioned between two annular grooves or along at least one annular groove 161 including a retainer 162 to thereby allow the retainer 162 to secure the first foam breaker device 200a in place along the second tubular connector 84. In some embodiments, an O-ring in the annual groove 161 can provide a frictional force against the inner passageway wall 208 of the first foam breaker device 200a to thereby prevent translational movement of the first foam breaker device 200a along the second tubular connector 84. Similarly, the impeller 85 and the second foam breaker device 200b can be securely coupled to the second tubular connector 84. Other attachment mechanisms and ways of coupling any number of foam breaker devices 200 along the second tubular connector 84 and/or drive shaft 72 are within the scope of this disclosure.

Furthermore, one or more of a variety of coupling features can be used to couple each foam breaker device 200 to the drive shaft 72 and/or second tubular connector 84. For example, a snap-fit and/or quick-connect engagement between complementing features of the foam breaker device 200 and the drive shaft 72 can allow the foam breaker device 200 to efficiently and effectively couple and/or uncouple (e.g., for reuse, cleaning, etc.) to a part of the drive shaft 72. In some embodiments, the foam breaker device 200 and/or drive shaft 72 can include barbed elements that can engage approximately any location along the drive shaft 72 for securing the foam breaker device 200 at a desired location. Other features for securing the foam breaker device 200 to a part of the drive shaft 72 are within the scope of this disclosure.

In some embodiments, the second tubular connector 84 can have a hexagonal cross-section that mates with a hexagonal cross-section of the passageways 205 of the foam breaker devices 200 such that rotation of the drive shaft 72 causes rotation of the second tubular connector 84 and the foam breaker devices 200. Similarly, the impeller 85 can be caused to rotate as a result of rotation of the second tubular connector.

As shown in FIG. 8, the third foam breaker device 200c can be coupled to a proximal end 90 of the second tubular connector 84. For example, the proximal end 90 can include a proximal end connector 91 that can couple to the distal connecting feature 350 of the third foam breaker device 200c. For example, the proximal end connector 91 can have a same or similar shape as the distal connecting feature 350, such as a square or hexagonal shape, that rotationally fixes the third foam breaker device 200c relative to the second tubular connector 84. For example, the proximal end connector 91 can receive a part of the distal connecting feature 350 and/or the distal connecting feature 350 can receive a part of the proximal end connector 91. In some embodiments, the proximal end connector 91 and/or the distal connecting feature 350 include tapering along a length that allows for rotationally fixed engagement of the proximal end connector 91 and distal connecting feature 350. Similarly, the proximal connecting feature 351 can be coupled with a distal end of a part of the drive shaft 72, such as a distal end of the tubular connector 80 of the impeller assembly 78, such that the third foam breaker device 200c rotates along with the drive shaft 72 and second tubular connector 84.

The foam breaker device 200 can be coupled to other parts of the fluid processing system 10 for reducing foam within the fluid processing system. For example, the foam breaker device 200 can be positioned in one or more containers 12, tubing, and/or ports (e.g., port 51 at FIG. 2). In some embodiments, one or more foam breaker devices 200 are coupled directly or indirectly to an embodiment of the drive shaft along which no impellers are coupled.

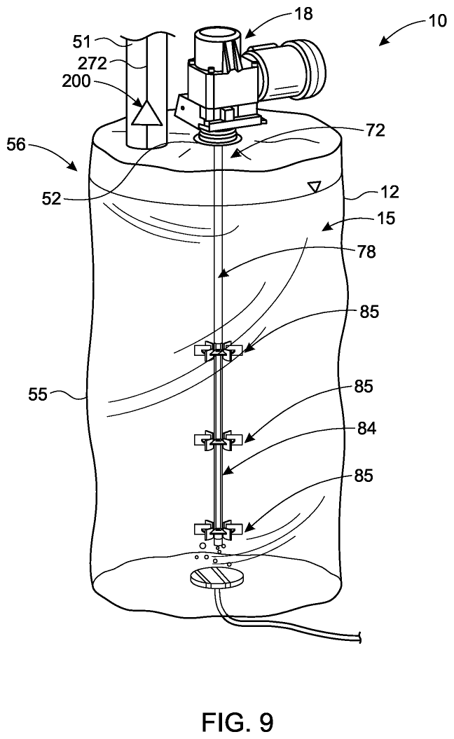

FIG. 9 illustrates an embodiment of the fluid processing system 10 including a port 51 (e.g., exhaust port) positioned along an upper end 56 of the container 12 that includes an embodiment of a first shaft 272 and a foam breaker device 200 coupled to the first shaft 272. In such a configuration, the foam breaker device 200 in the port 51 can prevent foam from leaving the inner chamber 15 of the container 12 such that foam does not come into contact with filters positioned outside of the inner chamber 15. For example, the foam breaker device 200 can rotate along the first shaft 272 and disrupt any foam that enters the port 51, thereby turning the foam into a liquid, which can then flow back into the inner chamber 15.

Although the port 51 having the first shaft 272 and foam breaker device 200 positioned therein is shown in FIG. 9 as being along a top or upper portion 56 of the container, the port 51 can be positioned along various locations along the side 55 and/or other parts of the container 12. The first shaft 272 along the port 51 can be driven by the same or different mixer system 18 including drive motor assembly 59 used to rotate the impellers 85 (e.g., via the drive shaft 72). The foam breaker device 200 can be positioned along a variety of tubing and types of containers 12 within the fluid processing system 10, such as to prevent foam from reaching filters. This can extend the life of such filters and eliminate or reduce the use of anti-foam.

The foam breaker device 200 can be positioned along a variety of drive shaft and/or impeller assembly 78 embodiments. For example, the foam breaker device 200 can be positioned along a flexible drive shaft, a rigid drive shaft, a foldable drive shaft, and/or a drive shaft having a ladder and/or helical configuration.

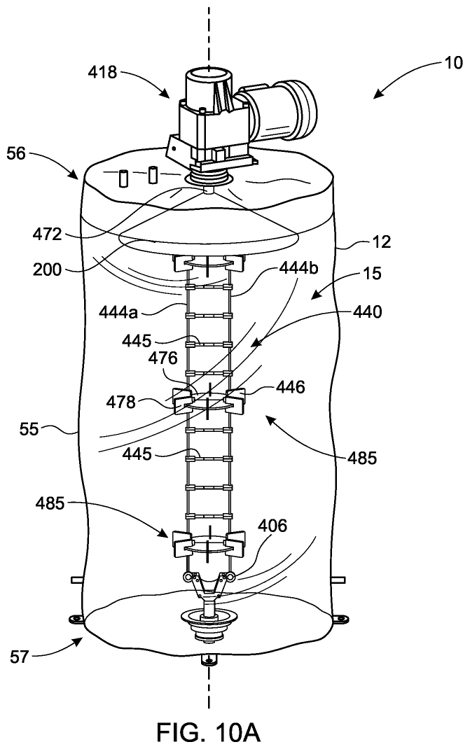

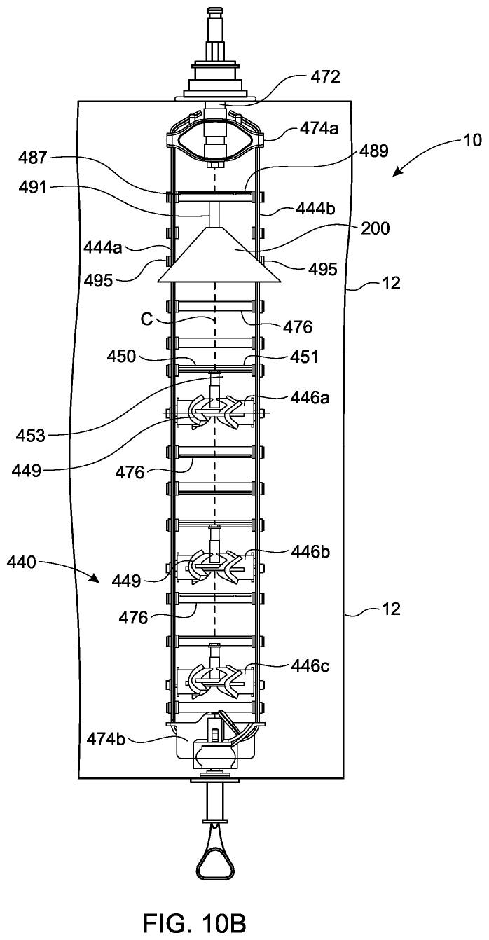

FIG. 10A illustrates an embodiment of a fluid processing system 10 including an embodiment of a mixer system 418 having a drive shaft 472 including a mixing assembly 440 having a ladder configuration. As shown in FIG. 10A, an embodiment of the foam breaker device 200 can be coupled to a part of the drive shaft 472 and/or mixing assembly 440. For example, the foam breaker device 200 can be positioned along the drive shaft 472 such that the foam breaker device 200 is positioned within the inner chamber 15 at or adjacent an upper end 56 of the container 12. In some embodiments, the foam breaker device 200 can be positioned at or adjacent a lower end 57 of the container 12 and/or in between the upper end 56 and lower end 57. For example, the foam breaker device 200 can be positioned along the inner chamber 15 such that the foam breaker device 200 is at a surface level of a liquid in the inner chamber 15. As such, varying volumes of liquid in the inner chamber 15 can result in the surface level of the liquid being at various positions along the container 12 and thus the foam breaker device 200 can be positioned at various positions along the drive shaft 472 and container 12 in order to be at or close to the surface level of the liquid. Further disclosure with regard to fluid processing system including mixing assembly 440 having a ladder configuration is disclosed in U.S. Pat. No. 11,352,598, granted Jun. 7, 2022, which is hereby incorporated by reference.

As shown in FIG. 10A, the mixing assembly 440 can include a plurality of mixing elements secured to drive lines 444a and 444b. In one embodiment, each mixing element can comprise an impeller 446 having a central hub 476 that spans between drive lines 444a and 444b and includes a plurality of blades 478 radially outwardly projecting therefrom. The impeller 446 can include a hub 476, which can have a variety of different configurations. For example, as depicted in FIG. 10, the hub 476 can comprise a tie 445 secured to drives lines 444a and 444b and include a flange outwardly projecting therefrom from which blades 478 project. In some embodiments, the tie 445 does not include blades 478 projecting therefrom. As shown in FIG. 10A, the drives lines 444a and 444b can couple to a support rod 406 at a lower end 57 of the container 12. Other configurations can also be used.

For example, rotation of the mixing assembly 440 can create a foam above the liquid in the inner chamber 15. The foam breaker device 200 can also rotate and disrupt the structure of the foam thereby turning the foam into liquid. As such, the foam breaker devices 200 described herein can be used in a variety of containers and with a variety of drive shafts to eliminate or reduce foam volume, foam formation, and the need for anti-foam.

FIG. 10B illustrates another embodiment of a fluid processing system 10 including an embodiment of a container 12 having the foam breaker device 200 mentioned above, coupled in-between the drive lines 444a and 444b of the mixing assembly 440. In this embodiment drive lines 444a, 444b extend between first retainer 474a and second retainer 474b, with the first retainer coupled to the drive shaft 472. Drive lines 444a, 444b are spaced apart by a plurality of rungs 476 interconnected between them. Rotation of the drive shaft 472, in turn, rotates drive lines 444a, 444b around a central axis C. Mixing assembly 440 can include a plurality of mixing elements secured to and extending between drive lines 444a and 444b at spaced apart locations along drive lines 444a and 444b. For example, each mixing element can comprise an impeller 446 including mixing blades 449. In the present example, mixing assembly 440 includes three impellers 446a, 446b, and 446c. For example, impeller 446a can be coupled between drive lines 444a and 444b by a first stabilizer 450 that can be secured to and between drive lines 444a and 444b. The first stabilizer 450 comprises a cross member 451 configured to securely engage with drive lines 444a, 444b, and rod 453 centrally projects from cross member 451 and is configured to be coupled to the impeller 446a. Similarly, other impellers 446b and 446c can be coupled between drive lines 444a, and 444b.

Similarly, to help stabilize foam breaker device 200 between drive members 444a and 444b, a second stabilizer 487 can be secured to and between drive members 444a and 444b and coupled with foam breaker device 200. Stabilizer 487 comprises a cross member 489 configured to securely engage with drive lines 444a, 444b, and rod 491 centrally projecting from cross member 489, which is configured to couple to foam breaker device 200. Although not required, rod 491 can have the same transverse cross section as passageway 205 of the mounting hub 204 for ease in coupling. Further drive lines 444a, and 444b are passing through an aperture 493 (not shown in FIG. 10B) in the wall structure of the foam breaker device with optionally a locking feature 495 locking the drive lines 444a, and 444b to the foam breaker device 200.

During assembly, stabilizer 487 is secured to and between drive members 444a and 444b and is coupled to foam breaker device 200, and the free end of rod 491 is received within passage 205 of the mounting hub 204. That is stabilizer 487 is secured to drive members 444a and 444b so that during operation, rod 491 cannot separate from foam breaker device 200. In some embodiments, the foam breaker device 200 can be positioned along the mixing assembly 440 such that the foam breaker device 200 is above or at least at a surface level of a liquid in the inner chamber 15.

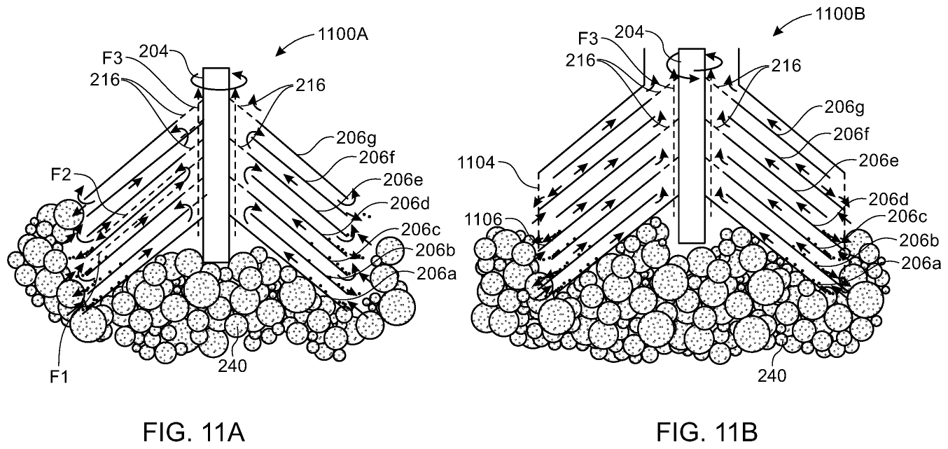

FIGS. 11A-11B illustrates an embodiment of foam breaker devices 1100A, 1100B including a coaxial stack of wall structures 206a-g extending circumferentially from the mounting hub 204. For example, the wall structures 206a-g can be coaxial and positioned along the mounting hub 204 such that the wall structures 206a-g are spaced apart by equal distances along the mounting hub 204. In other examples, the wall structures 206a-g can be spaced apart by unequal distances along the mounting hub 204, including the formation of sections of two or three wall structures. The section of wall structures can be spaced apart at variable distances. As previously mentioned, the mounting hub 204 including the stack of wall structures can be coupled to the second tubular connector 84 (FIG. 2) that can be coupled directly or indirectly to the drive shaft for rotation of the foam breaker devices 1100A, 1100B in a bioreactor, fermenter, or any suitable bioprocessing equipment. Alternatively, the foam breaker devices 1100A, 1100B can be positioned in the exhaust port 51 attached to a bioreactor, so that the exhaust gases are free of particles, debris, and condensate.

Referring to FIG. 11A, the foam breaker device 1100A, can include wall structures 206a-g having a fin structure 213 as shown in FIG. 4J or wall structures without having a fin structure as shown in FIG. 4H or FIG. 4I. The wall structures can be arranged coaxially such that the apertures 216 are aligned with each other. Other arrangements with the apertures 216 not aligned with each other also fall under the scope of this disclosure. For example, each of the wall structures 206a-g can be made of rigid material. In other examples, each of the wall structures 206a-g can be made of silicone rubber or any other material, including polyethylene (PE), polypropylene (PP), polycarbonate (PC), acrylonitrile Butadiene styrene (ABS), 316 grade stainless steel (316ss), or any other molded, machined, or printed material.

Upon positioning the foam breaker device 1100A, in a surrounding including foam 240 as shown in FIG. 11A and rotating the foam breaker device 1100A in a clockwise direction (for example relative to the arrangement of fin structures 213 shown in FIG. 4J), the foam 240 gets pulled from the edge portion into a spacing between the wall structures along a direction F1 towards the mounting hub 204. Movement of the foam 240 in spacings between the wall structures 206a-g combined with the continuous rotation of the foam breaker device 1100A results in the foam 240 being disintegrated into gas, light particle phase, heavy particles phase, water, and debris. Owing to a centrifugal force generated by the rotation of the foam breaker device 1100A the heavy particles phase, water, and debris fall back into an adjacent spacing along a second direction F2 and back to the bioreactor container 12. During this process, gaseous matter released is allowed to move out of the apertures 216 along a third direction F3, and further to an exhaust system. Gaseous matter released out from the foam breaker device 1100A is free of particulate matter and hence reduces clogging in the exhaust filters.

Upon rotation of the foam breaker device 1100A in an anticlockwise direction relative to the arrangement of fin structures as shown in FIG. 4J, the foam 240 gets pulled toward the center of the mounting hub 204 (in direction F1) and around the mounting hub 204, and the foam 240 moves from center to the edge portion through a spacing between the wall structures 206a-g along a direction F2. As mentioned above during the movement of the foam 240 in the spacing between the foam breakers in directions F1 and/or F2 the foam can be disintegrated into gaseous matter, light particle phase, heavy particles phase, water, and debris. The gaseous matter is allowed to move out of the apertures 216 and along a third direction F3, and further to an exhaust system. The heavy particles phase, water, and debris fall back into an adjacent spacing along a second direction F2 and back to the bioreactor container 12.

Referring to FIG. 11B, the foam breaker device 1100B, can include wall structures 206a-g having a fin structure as shown in FIG. 4J or wall structures without having a fin structure as shown in FIG. 4H or FIG. 4I. The wall structures can be arranged coaxially such that the apertures 216 are aligned with each other. A first set of alternate wall structures 206a, 206c, 206e, and 206g extend from the mounting hub 204, and a second set of alternate wall structures 206b, 206d, 206f are attached to walls 1104 of port 51 on the bioreactor. Further, the second set of alternate wall structures 206b, 206d, and 206f include perforation 1106 near the edge of the foam breakers for movement of foam, gas, light particle phase, heavy particles phase, water, and debris. In this case, the wall structures can be made of rigid materials as mentioned above.

Upon positioning the foam breaker device 1100B, in a surrounding including foam 240 as shown in FIG. 11B, and rotating the foam breaker device 1100B in a clockwise or anticlockwise direction relative to the arrangement of fin structures 213 (shown in FIG. 4J), the first set of alternate wall structures 206a, 206c, 206e, and 206g are rotating, while the second set of alternate wall structures 206b, 206d, 206f remain stationary. The foam 240 which enters the foam breaker 1100B from the edges or from the center, moves continuously along spacings between the wall structures 206a-g and gets disintegrated to release gaseous matter free of particles. The gaseous matter is allowed to move out of the foam breaker device 1100B in a direction F3.

Additionally, in the embodiments described above the mounting hub 204 in foam breaker devices 1100A, 1100B can have perforations along its wall to receive foam and push the foam into spacings between the wall structures 206a-g such that excessive foam produced in container 12 during bioprocessing can be effectively disintegrated and gaseous matter free of particulate matter and condensate can be released out of the container 12.

While the invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. Therefore, the invention in its broadest aspects is not limited to the specific details shown and described. The various features disclosed herein may be used in any combination necessary or desired for a particular application. Consequently, departures may be made from the details described herein without departing from the spirit and scope of the claims which follow.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C,” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A foam breaker device of a fluid processing system comprising:
a mounting hub extending along a first axis of the foam breaker device, the mounting hub configured to rotatably couple to a drive motor assembly; and
a wall structure extending circumferentially from the mounting hub at an angle relative to the first axis of the foam breaker device, the wall structure causing a reduction in a foam volume as a result of at least a part of the wall structure contacting the foam volume.

2. The foam breaker device of claim 1, wherein the wall structure is formed of one or more of a flexible material and a foldable material.

3. The foam breaker device of claim 1, wherein the wall structure is formed of one or more of a hydrophilic material and a hydrophobic material.

4. The foam breaker device of claim 1, further comprising a macro texture or micro-texture on an inner wall surface or an outer wall surface of the wall structure.

5. The foam breaker device of claim 1, wherein the wall structure comprises two or more overlapping fin structures.

6. The foam breaker device of claim 1, wherein the wall structure comprises a plurality of radially extending bristle structures.

7. The foam breaker device of claim 1, wherein the angle is approximately between 10 degrees and 90 degrees.

8. The foam breaker device of claim 1, wherein the wall structure is adjustable between an extended configuration comprising a first outer diameter and a collapsed configuration comprising a second outer diameter that is smaller than the first outer diameter.

9. The foam breaker device of claim 1, further comprising a coupling feature on the mounting hub and configured to couple the mounting hub to a first shaft.

10. The foam breaker device of claim 9, wherein the coupling feature further comprises a passageway extending along the mounting hub, the passageway configured to receive the first shaft therein.

11. The foam breaker device of claim 9, wherein the coupling feature is shaped to have a sliding translational engagement with the first shaft.

12. The foam breaker device of claim 9, wherein the coupling feature is rotationally fixed relative to the first shaft when the foam breaker device is coupled to the first shaft such that rotation of the first shaft causes the foam breaker device to rotate about the first axis.

13. The foam breaker device of claim 1, wherein the foam breaker device rotates at a speed of approximately 50 rotations per minute (RPMs) to approximately 1200 RPMs.

14. The foam breaker device of claim 1, wherein the wall structure comprises a through hole positioned adjacent the mounting hub for allowing one or more of a liquid and gas to pass through the through hole.

15. The foam breaker device of claim 14, wherein the liquid is a cell culture medium.

16. A fluid processing system, comprising:
a container comprising an inner chamber configured to contain a liquid for processing; and
a foam breaker device as in any one of claims 1-8.

17. The fluid processing system of claim 16, further comprising a drive shaft that extends into the inner chamber of the container for assisting with mixing the liquid, the foam breaker device being coupled to the drive shaft.

18. The fluid processing system of claim 17, wherein the drive shaft comprises one or more of a flexible drive shaft, a rigid drive shaft, a foldable drive shaft, and the drive shaft having a ladder and/or helical configuration.

19. The fluid processing system of claim 17, wherein the foam breaker device is coupled to the drive shaft in a first orientation relative to the drive shaft, wherein the first orientation comprises an inner surface of the foam breaker device directed toward a distal end of the drive shaft and/or a lower end of the container.

20. The fluid processing system of claim 17, wherein the foam breaker device is coupled to the drive shaft in a second orientation relative to the drive shaft, wherein the second orientation comprises the inner surface of the foam breaker device directed toward a proximal end of the drive shaft and/or upper end of the container.

21. The fluid processing system of claim 16, wherein the container comprises one or more of a bioreactor, a flexible bag, a condenser, a fermenter, a mixing chamber, and a tubing.

22. The fluid processing system of claim 16, wherein the foam breaker device is positioned along a port in fluid communication with an inner chamber of the container.

23. A method of processing a liquid in a fluid processing system, comprising:
receiving a liquid in an inner chamber of a container of the fluid processing system;
rotating a drive shaft extending in the inner chamber for mixing the liquid; and
rotating a foam breaker device to reduce a foam volume in the inner chamber, the foam breaker device comprising any one of claims 1-8.

24. The method of claim 23, wherein the liquid comprises one or more of human cells, microorganisms, bacteria, fungi, algae, plant cells, animal cells, protozoans, and nematodes.

25. The method of claim 23, wherein the container comprises one or more of a bioreactor, a flexible bag, a condenser, a fermenter, a mixing chamber.

26. The foam breaker device of claim 23, wherein a perimeter of the wall structure comprises a diameter that is greater than three-quarters of an inner diameter of the inner chamber of the container.

27. The foam breaker device of claim 23, wherein a ratio of a diameter of the foam breaker device to an inner diameter of the inner chamber of the container is 1:2.

28. A method of assembling a fluid processing system, comprising:
coupling a mixing system to a container of the fluid processing system;
coupling a foam breaker device to a drive shaft of the mixing system, the foam breaker device, comprising:
a mounting hub extending along a first axis of the foam breaker device, the mounting hub comprising a coupling feature for coupling the foam breaker device to the drive shaft; and
a cone shaped wall extending circumferentially from the mounting hub at an angle relative to the first axis of the foam breaker device, the cone shaped wall reducing a foam as a result of at least a part of the cone shaped wall contacting the foam.

29. The method of claim 28, wherein the coupling feature slidably translates along the drive shaft.

30. The method of claim 28, wherein the coupling comprises coupling the coupling feature of the mounting hub to the drive shaft and securing the foam breaker device at a position along the drive shaft.

31. The method of claim 28, wherein the coupling feature is rotationally fixed relative to the drive shaft when coupled thereto such that rotation of the drive shaft causes rotation of the foam breaker device.

32. The method of claim 28, wherein the coupling feature comprises a square or hexagonal cross-section.

33. The method of claim 28, wherein the foam breaker device is coupled to the drive shaft in a first orientation relative to the drive shaft, wherein an inner surface of the cone shaped wall is directed toward a distal end of the drive shaft and/or a lower end of the container.

34. The method of claim 28, wherein the foam breaker device is coupled to the drive shaft in a second orientation relative to the drive shaft, wherein an inner surface of the cone shaped wall is directed toward a proximal end of the drive shaft and/or upper end of the container.

35. The method of claim 28, further comprising:
coupling more than one foam breaker device to the drive shaft.

36. The method of claim 28, wherein the drive shaft extends along the container, the container comprising one or more of a bioreactor, a flexible bag, a condenser, a fermenter, a mixing chamber, and a tubing.

37. The method of claim 28, wherein the foam breaker device is positioned along a port in fluid communication with an inner chamber of the container.

38. The method of claim 28, wherein the drive shaft comprises one or more of a flexible drive shaft, a rigid drive shaft, a foldable drive shaft, and the drive shaft having a ladder and/or helical configuration.

39. A method of suppressing foam in a fluid processing system, comprising rotating a drive shaft extending in a inner chamber containing a liquid; and
rotating a foam breaker device to reduce and/or prevent formation of a foam in the inner chamber, the foam breaker device comprising any one of claims 1-8.

40. A method of processing a liquid in a fluid processing system, comprising receiving a liquid in an inner chamber of container of the fluid processing system; and
rotating a drive shaft extending in the inner chamber for mixing the liquid; and
rotating a foam breaker device to reduce a foam volume in the inner chamber, the foam breaker device comprising any one of claims 1-8.

41. The method of claim 40, wherein the foam breaker device is positioned above the liquid.

42. The method of claim 40, wherein the container comprises one or more of a bioreactor, a flexible bag, a condenser, a fermenter, a mixing chamber.

43. The method of claim 40, wherein the liquid comprises one or more of human cells, microorganisms, bacteria, fungi, algae, plant cells, animal cells, protozoans, and nematodes.

44. A method of reducing foam in a container of a bioprocessing system, comprising:
a foam breaker device comprising a mounting hub extending along a first axis of the foam breaker device, the mounting hub rotatably coupled to a drive motor assembly, and a wall structure extending circumferentially from the mounting hub;
rotating the foam breaker device at a first speed to extend foam breaker device from a first configuration to a second configuration;
rotating the foam breaker device at a second speed; and
contacting the foam by at least a portion of the wall structure of the foam breaker device to reduce foam in the container.

45. The method of claim 44, wherein the wall structure of the foam breaker device in the second configuration is aligned with the first axis at an angle ranging between 10-90 degrees.

46. The method of claim 44, wherein the first speed is less than the second speed.

47. The method of claim 44 wherein the second speed ranges between 140-160 inches/second.

48. A method of reducing foam in a container of a bioprocessing system, comprising:
a foam breaker device comprising a mounting hub extending along a first axis of the foam breaker device, the mounting hub rotatably coupled to a drive motor assembly, and a wall structure extending circumferentially from the mounting hub;
holding the foam breaker device in a first configuration;
releasing the foam breaker device to extend the wall structure of the foam breaker device from the first configuration to a second configuration;
maintaining the foam breaker device at the second configuration; and
rotating the foam breaker device having the second configuration to cause at least a portion of the wall structure of the foam breaker device to continuously contact the foam to reduce the foam in the container.

49. The method of claim 48, wherein the foam breaker device includes a memory metallic wire to substantially support the wall structure.

50. The method of claim 48, wherein the foam breaker device includes a memory metallic wire embedded in the wall structure.

51. The method of claim 48, wherein the wall structure of the foam breaker device in the second configuration is aligned with the first axis at an angle ranging between 10-90 degrees.

52. A foam breaker device of a fluid processing system comprising,
a mounting hub extending along a first axis of the foam breaker device, the mounting hub configured to rotatably couple to a drive motor assembly; and
a plurality of wall structures extending circumferentially from the mounting hub at an angle relative to the first axis of the foam breaker device, each of the plurality of wall structures causing a reduction in a foam volume as a result of at least a part of each of the plurality of wall structures contacting the foam volume.

53. The foam breaker device of claim 52, wherein each of the plurality of wall structures comprise a plurality of fin structures radially extending outward from the mounting hub, and the plurality of fin structures being arranged in a clockwise or anti-clockwise direction.
